(19) United States
(12) Patent Application Publication
(10) Pub. No.: US 2006/0020266 A1
(43) Pub. Date: Jan. 26, 2006

(54) Bioabsorbable implant

(76) Inventor: John Joseph Cooper, Cheshier (GB)

Correspondence Address: SMITH-HILL AND BEDELL, P.C., 16100 NW CORNELL ROAD, SUITE 220, BEAVERTON, OR, 97006, US

(21) Appl. No.: 10/536,273

(22) PCT Filed: Jan. 27, 2004
(86) PCT No.: PCT/GB04/00326

§ 371 Date: May 24, 2005

(30) Foreign Application Priority Data
Jan. 29, 2003 (GB) 0302026.0

(51) Int. Cl. A61F 2/30

(52) U.S. Cl. 606/77

(57) ABSTRACT

A method of forming a bioabsorbable implant, the method comprising forming an implant from a composite of a bioabsorbable polymer and a bioactive ceramic filler, and abrading the surface of the implant with a biocompatible abrasive material such as a hydroxyapatite grit, such that part of the outer surface of the implant is provided by the ceramic filler.

This invention relates to bioabsorbable implants and also methods of making bioabsorbable implants.

Metallic implants have been used successfully for a wide range of tissue fixation applications in orthopaedic and maxillofacial surgery. Metals such as stainless steel and titanium alloy have been used since they have good mechanical strength and are relatively bioinert.

However, the presence of these materials inside the body can make MRI imaging of the site impossible, can give long term problems of metal ion release, can result in stress shielding effects due to their high modulus with resulting bone resorption around the implant and can often result in further surgery to remove the implants.

In recent years there has been an increasing interest in the use of bioabsorbable polymers to replace metallic implants in a number of orthopaedic and maxillofacial fixation applications. The advent of synthetic bioabsorbable polymers and their use in a range of indications can overcome many of the problems associated with metallic implants. These materials are bio-absorbed slowly in the body first losing strength and then mass, thus slowly transferring mechanical support to the healing tissue and negating the need for further surgical intervention to remove the device.

The synthetic bioabsorbable polymers including poly lactide, poly glycolide, poly dioxanone, poly caprolactone, poly hydroxybutyrate and poly hydroxvalerate, while offering many advantages over metallic implants in certain indications, do have some limitations and drawbacks. Their modulus is generally less than that of the bone which they can be used to support. This can lead to macromotion at a fracture site when loaded and consequently inhibition of bone healing. These materials have no osteoconductive potential and hence no potential to bond with adjacent bone or to be replaced by new bone once fully resorbed. Additionally they have been shown to be susceptible to a mechanism known as autocatalytic degradation whereby the formation of the acidic by-products of hydrolysis of the polymer results in a lowering of pH within the implant. This accelerates the rate of further degradation and results in acidosis and the potential for the clinical condition of weeping sinus or sterile abscess formation.

These limitations and drawbacks of the synthetic bioabsorbable polymers cited above can be alleviated by incorporating into the polymer matrix a bioactive, i.e. osteoconductive, ceramic such as hydroxyapatite, tri-calcium phosphate or bioactive glass. These materials in powdered form confer a number of advantages to the polymer. Their presence as a filler increases modulus such that modulus matching to bone becomes possible. They are osteoconductive, which is a significant advantage at or in a bony site. They add a degree of X-ray opacity to the X-ray transparent polymer thus making visualisation a little easier and they can also provide a buffering effect to the acidic degradation products of the polymer.

An additional significant deficiency of the synthetic bioabsorbable polymers particularly when they are used as bony site fixation and support applications or as scaffolds for tissue engineering applications is their hydrophobicity. They are not wetted by aqueous fluids and as such, cellular attachment and subsequent proliferation is inhibited which limits their potential for early tissue regeneration.

Traditional techniques for forming devices which consist of a powder filled thermoplastic polymer involve the melt blending of the components followed by extrusion, injection moulding or compression moulding. These techniques are designed to provide a homogeneous distribution of the filler particles throughout the polymer matrix. Injection moulding is particularly suited to the cost effective, mass production of complex shaped components with a minimum of post-moulding finishing.

However, the surface of melt moulded composite devices produced by conventional forming techniques such as those described above, invariably consists solely of the polymer component. Each and every filler particle becomes surrounded by polymer during melt blending and no particles are freely exposed at the surface of the moulded device. Advantageous direct contact between the bioactive ceramic particles and the adjacent body tissue can only occur weeks or months after implantation when bioabsorption of the polymer surface layer has proceeded so far as to develop cracks or crazes which expose the sub-surface particles. The presence of exposed bioactive particles in the surface of such implants improves their hydrophilicity, biocompatability and osteoconductive potential and enables enhanced cellular attachment and proliferation and early biological incorporation.

To provide these beneficial features it would therefore be necessary to remove the polymer surface layer of such melt-moulded devices thus exposing the sub surface particles. This could be achieved through surface machining or grinding. Such surface machining processes are, however, difficult and time consuming particularly for complex shaped devices such as bone screws, plates, tacks or spinal spacers. Another option would be to mould a blank part utilising the desired composite material and machining from this blank the finished implant device. This process, however, would not be efficient or cost effective for mass produced devices and is wasteful of expensive material.

Alternative processes of surface cleaning, polishing or abrasion by grit blasting or bead blasting utilise grit consisting of such abrasive materials as corundum, silicon carbide or glass beads. However, invariably, a proportion of these materials would become stuck to or embedded in the surface of the implant device desired to be cleaned, polished or abraded. For bioabsorbable implant devices the presence of such abrasive foreign body materials would be highly undesired and totally unacceptable. Furthermore, techniques such as grit blasting are line-of-sight processes, and cannot therefore be used to treat re-entrant surfaces.

According to the present invention there is provided a method of forming a bioabsorbable implant, the method including forming an implant member from a composite of a bioabsorbable polymer and a bioactive ceramic filler, and abrading the surface of the implant member with a biocompatible abrasive material such that part of the outer surface of the implant member is provided by the ceramic filler, to form a usable implant.

The implant member is preferably formed by any of injection moulding, compression moulding or extrusion.

The biocompatible abrasive material may comprise a bioactive ceramic powder which may be hydroxyapatite or tricalcium phosphate. Alternatively, the biocompatible abrasive material may comprise a soluble biocompatible salt, which may be sodium chloride.

Following abrasion, the biocompatible abrasive material is preferably separated from the implant member. Where the biocompatible abrasive material is a ceramic powder, separation is preferably carried out by screening. Where the biocompatible abrasive material is a soluble biocompatible salt, the separation may be carried out by rinsing with water.

The abrasive material preferably has a particle size of between 10 and 1000 microns, and desirably between 30 and 500 microns.

The abrasion may be carried out by tumbling, shaking or vibrating the implant member together with the abrasive material, which may take place in a closed container.

The invention also provides a bioabsorbable implant, the implant comprising a composite of a bioabsorbable polymer and a bioactive ceramic filler, with some of the outer surface of the implant being provided by the ceramic filler.

The bioabsorbable polymer may comprise any of poly lactide, poly glycolide, poly dioxanone, poly caprolactone, poly hydroxybutyrate or poly hydroxvalerate, copolymers thereof and/or mixtures thereof.

The bioactive ceramic filler may comprise any of hydroxyapatite, tri-calcium phosphate, calcium sulphate or bioactive glass.

The implant may be in the form of a screw, a spinal interbody fusion device, pin, plate, tack, suture, wound care patch, osteotomy wedge or other item usable in surgery.

Embodiments of the present invention will now be described by way of example only.

### EXAMPLE 1

A hydroxyapatite grit was prepared as follows:—A high surface area hydroxyapatite powder i.e. a powder with inherent sinterability, was added to water with stirring to form a slurry. The powder suspension was de-watered on a Buchner filter and subsequently dried in an oven at 120° C. to form a cake. This was subsequently pre-fired at 900° C. with 1 hour hold at peak temperature. On cooling the pre-sintered hydroxyapatite cake was crushed using a pestle and mortar and sieved to pass a 350 micron mesh sieve. The material was then sieved to remove sub 250 micron sized particles. The resulting angular shaped particles which had a sieve size range of 250-350 microns were then sintered by firing to a temperature of 1200° C. with 2 hours hold at peak temperature. When cool the resulting angular hydroxyapatite grit was used as an abrasive to remove the surface of injection moulded implant devices.

Bone fixation screws were injection moulded using a composite mixture of poly lactide and hydroxyapatite in the proportions of 70:30 parts by weight. A batch of 100 such screws together with 1 kg of the above hydroxyapatite grit was charged into a 2.5 litre capacity jar which was lidded and subsequently rotated at 50 rpm for a period of 6 hours. At the completion of this tumbling action the screws and grit were removed from the jar and separated by shaking onto a 2 mm mesh sieve which allowed passage of the grit but retained the screws. Close examination of the surface of the screws showed them to be abraded and hydroxyapatite filler particles were exposed in the surface. By this means an implant device was produced which had a surface structure amenable to early cellular attachment on implantation and a potentially more rapid biological incorporation.

### EXAMPLE 2

A tri-calcium phosphate grit was prepared as follows:—A high surface area tri-calcium phosphate powder, i.e. a powder with inherent sinterability, was added to water with stirring together with a percent of organic binder such as PVA to form a slurry. This powder suspension was spray dried to form rounded granules with a particle size in the range 30-60 microns. This powder was then sintered by firing to 1100° C. with 2 hours hold at peak.

When cold the resultant free flowing tri-calcium phosphate grit was used as an abrasive to remove the polymer surface layer from bioabsorbable composite interbody fusion devices. These devices were injection moulded from a mixture of poly L-lactic acid and hydroxyapatite powder in the proportions of 75:25 parts by weight and are designed to be inserted between adjacent vertebrae to restore and maintain disc height in spinal fusion surgery.

A batch of 50 such devices were charged into a 1 gallon capacity jar together with 2 kg of the tri-calcium phosphate grit. This was then shaken vigorously in a vibration mill for 15 minutes. The charge was then removed from the mill and the devices were separated from the grit by shaking on a sieve which allowed the passage of the grit but retained the devices. Close examination of the devices revealed that their surfaces were roughened or abraded to expose hydroxyapatite particles. This was true not only of the outer surfaces of the devices, but also of the inner re-entrant surfaces which are designed to contain a bone graft material and would be difficult to abrade using prior art techniques. The nature of the surfaces of this device enables a more rapid and thorough osseointegration and biological acceptance than similarly shaped devices not containing these unique features.

### EXAMPLE 3

A sodium chloride grit was prepared by crushing and sieving rock salt to give a size fraction of 250-500 microns. This was used as the abrasive grit to remove the surface polymer film from batches of bioabsorbable composite implant devices by similar methods to those described in the two preceding examples. Following the abrading step the devices were separated from the salt and excess salt was removed by rinsing the devices in sterile water followed by drying at 37° C.

There are thus described bioabsorbable implants and methods for making them which provide for significant advantages relative to the prior arrangements outlined in the introduction to the specification. Conventional methods are used for making the implant member. The abrasive material is made and used with relatively conventional methods thereby providing an inexpensive process for providing implants with significantly increased performance and advantages. With the present invention it is possible to treat re-entrant surfaces, which is not possible with most existing processes, as detailed above.

Various modifications may be made without departing from the scope of the invention. For instance other bloactive or biocompatible materials could be used in these methods as outlined above, and the abrasive materials could be produced by other methods. The abrading may be carried out differently, and may be carried out in open container, which container may be rotated.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-22. (canceled)

23. A method of forming a bioabsorbable implant, the method comprising forming an implant member from a composite of a bioabsorbable polymer and a bioactive ceramic filler, characterized in that the method also includes abrading the surface of the implant member with a biocompatible abrasive material such that part of the outer surface of the implant member is provided by the ceramic filler, to form a usable implant.

24. A method according to claim 23, wherein the implant member is formed by injection moulding.

25. A method according to claim 23, wherein the implant member is formed by compression moulding.

26. A method according to claim 23, wherein the implant member is formed by extrusion.

27. A method according to claim 23, wherein the biocompatible abrasive material comprises a bioactive ceramic powder.

28. A method according to claim 27, wherein the bioactive ceramic powder is hydroxyapatite or tricalcium phosphate.

29. A method according to claim 23, wherein the biocompatible abrasive material comprises a soluble biocompatible salt.

30. A method according to claim 29, wherein the soluble biocompatible salt is sodium chloride.

31. A method according to claim 23, wherein following abrasion, the biocompatible abrasive material is separated from the implant member.

32. A method according to claim 31, wherein the biocompatible abrasive material comprises a bioactive ceramic powder and the separation is carried out by screening.

33. A method according to claim 31, wherein the biocompatible abrasive material comprises a soluble biocompatible salt and the separation is carried out by rinsing with water.

34. A method according to claim 23, wherein the abrasive material has a particle size of between 10 and 1000 microns.

35. A method according to claim 34, wherein the abrasive material has a particle size of between 30 and 500 microns.

36. A method according to claim 23, wherein the abrasion is carried out by tumbling, shaking or vibrating the implant member together with the abrasive material.

37. A method according to claim 36, wherein the tumbling, shaking or vibrating of the implant member together with the abrasive material takes place in a closed container.

38. A bioabsorbable implant, the implant comprising a composite of a bioabsorbable polymer and a bioactive ceramic filler, wherein some of the outer surface of the implant is provided by the ceramic filler.

39. A bioabsorbable implant according to claim 38, wherein the bioabsorbable polymer comprises any of poly lactide, poly glycolide, poly dioxanone, poly caprolactone, poly hydroxybutyrate or poly hydroxvalerate, copolymers thereof and/or mixtures thereof.

40. A bioabsorbable implant according to claim 38, wherein the bioactive ceramic filler comprises any of hydroxyapatite, tri-calcium phosphate, calcium sulphate or bioactive glass.

41. A bioabsorbable implant according to claim 38, wherein the implant is in the form of a screw, a spinal interbody fusion device, pin, plate, tack, suture, wound care patch, osteotomy wedge or other item usable in surgery.
